(19) United States
(12) Patent Application Publication
(10) Pub. No.: US 2015/0275295 A1
(43) Pub. Date: Oct. 1, 2015

(54) ASSAY FOR THE PARALLEL DETECTION OF BIOLOGICAL MATERIAL BASED ON PCR

(71) Applicant: NATIONAL UNIVERSITY OF SINGAPORE, Singapore (SG)

(72) Inventors: Linfa Wang, Singapore (SG); Eng Eong Ooi, Singapore (SG); October Michael Sessions, Singapore (SG); Danielle Elizabeth Anderson, Singapore (SG)

(21) Appl. No.: 14/437,592

(22) PCT Filed: Oct. 22, 2013
(86) PCT No.: PCT/SG2013/000455

§ 371 Date: Apr. 22, 2015

(30) Foreign Application Priority Data
Oct. 22, 2012 (GB) 1218909.8

(51) Int. Cl. C12Q 1/68; C07K 19/00

(52) U.S. Cl. CPC C12Q 1/6876; C07K 19/00; C12Q 2600/16

(57) ABSTRACT

The invention concerns a novel parallel method for detecting biological material, in particular peptides or proteins, in a sample at least one probe for use in the said method, a plurality, or library, of said probes for use in said method, and a kit of parts for carrying out said method wherein said probe comprises a binding partner that is specific for said peptide or protein and, attached thereto, an oligonucleotide comprising: i) a first sequence that is complementary to a forward primer sequence for amplification of said oligonucleotide; ii) a second sequence that is complementary to a reverse primer sequence for amplification of said oligonucleotide; and iii) positioned between said first and second sequences an identification sequence of nucleotides or barcode.

The invention relates to a novel method for detecting at least one target molecule(s) in a sample, in particular for detecting peptides, proteins, lipids or carbohydrate in a sample; at least one probe for use in the said method; a plurality, or library, of said probes for use in said method; and a kit of parts for carrying out said method.

### BACKGROUND OF INVENTION

Molecular interactions involving, for example, peptides or proteins (e.g., antibody-antigen interactions, hormone-receptor interactions, virus-receptor interactions, enzyme-substrate interactions, to name but a few) represent the most complex and important processes in any biological system. Their detection can provide valuable information concerning the status of the system and so can provide important information of diagnostic, therapeutic or commercial value. For example, the following is a non-exhaustive list of the sorts of information that can be derived by monitoring peptide or protein interactions.
a) Diagnosis of respiratory infectious diseases of unknown causes
b) Investigation of CNS infection of unknown etiology
c) Investigation of autoimmune diseases
d) Investigation of cancer with a potential link to infectious agents
e) Investigation of major chronic diseases such as multiple sclerosis, diabetes mellitus, obesity and other metabolic syndromes, Crohn's disease and ulcerative colitis, etc.
f) Biomarker discovery for any human medical condition

However, unlike nucleic acid-based detection, there is currently no effective methodology for the amplification of peptides, proteins, lipids or carbohydrate-based interactions in a biological system. This means, especially where quantity is low, many interactions go unrecognised or undetected. Specific amplification of such interactions or signals is needed to provide a suitably sensitive method. Such a method would have significant impact in all areas of biological and medical research. The latter includes, but is not limited to: characterization of antibody-mediated immune responses for diagnostic and vaccine related use; screening for protein-protein interactions in biological processes or cellular signalling; screening of drug-protein binding or interaction, such as off-target or non-specific binding that could lead to side effects; screening for protein-glycoprotein binding, such as identification of virus-receptor binding for cellular entry; the characterization of post-translational glycan and oligosaccharide modifications on proteins for characterization and development of biologic drugs; and screening for protein-phospholipid interaction in biological processes such as determining how blood clotting proteins bind to cellular membranes.

In the area of monitoring antibody-mediated immune responses, being able to monitor peptide or protein-based signals in a biological system would be of great value. For example, any disease involving an infectious agent will very likely produce a specific host response against that agent. This includes conditions such as encephalitis resulting from infectious agents that are presently difficult to diagnose. Additionally, antibody-mediated immune responses from non-infectious diseases such as cancer, autoimmune diseases and chronic fatigue syndrome also represent areas likely to benefit from monitoring a peptide or protein-based signal in a biological system.

Whilst it is currently possible to monitor peptides or proteins in response to an antibody-mediated immune response, most of the current technologies for peptide or protein-based detection only allow for examination of immune responses against a single or small number of targeted agents. This makes the interrogation of a system repetitive, laborious, time consuming and expensive. These drawbacks could be overcome if it was possible to provide a high-throughput peptide or protein-based screening method, or microarray, for antibody monitoring. Whilst high-throughput peptide-based microarrays for antibody monitoring have been reported, these are not widely used due to their low sensitivity and lack of reproducibility (H. Andresen and C. Grötzinger (2009) Deciphering the antibodyome-peptide arrays for serum antibody biomarker diagnostics. Current Proteomics, 2009, 6, 1-12).

The invention described herein thus aims to overcome the disadvantages associated with the prior art.

Statements of Invention

According to a first aspect of the invention there is provided a probe for detecting and/or quantifying at least one target molecule(s) in a sample comprising:
a) at least one binding partner that is specific for said target molecule(s) and, attached thereto;
b) an oligonucleotide wherein said oligonucleotide comprises:
   i) a first sequence that is complementary to a forward primer sequence for amplification of said oligonucleotide;
   ii) a second sequence that is complementary to a reverse primer sequence for amplification of said oligonucleotide; and
   iii) positioned between said first and second sequences an identification sequence of nucleotides or Barcode wherein said Barcode acts an indicator for said target molecule(s) and consists of a certain number of nucleotides arranged in a unique order and further wherein the number of unique arrangements of said nucleotides provided by the number and nature of said nucleotides is greater than the number of target molecules in said sample.

Reference herein to a binding partner that is specific for said target molecule(s) means the binding partner is able to bind to said target molecule(s) to the exclusion of binding with other target molecule(s) of either a different or similar nature and, indeed, in some instances is unable to bind with any other target molecule(s).

In a preferred embodiment of the invention a plurality of probes may be provided as a probe library, once new probes are developed this library may be expanded and, additionally, or alternatively, said library may also be customized for a particular purpose such as, without limitation, hospital-based diagnosis such as for example, the diagnosis of acute respiratory infections, where approximately 100 probes may be needed. The expanded library may, however, comprise 10⁵ or 10⁶ probes and when of this size it is expected to pick up mimitopes (epitopes mimicking the original native epitopes), this will make the library extremely powerful and useful for certain applications, such as the investigation of cross-reactive antigens for autoimmune diseases and biomarker discovery.

In a preferred embodiment of the invention said binding partner has at least one epitope that is specific for said target molecule(s) but, ideally, it has a plurality of epitopes that are specific for said target molecule(s).

Most preferably, said binding partner comprises at least one and, ideally, a plurality of peptides and/or proteins which, individually or collectively, comprise at least one and, preferably, a plurality of epitopes that are specific for said peptide or protein to be detected.

In a further preferred embodiment of the invention said probe is further provided with a tag or label that facilitates the identification of same in a multiplex assay. Tags or labels of this sort are characterised by being amplifiable by PCR and so, ideally, comprises a further short DNA sequence of a distinctive nature that is, preferably, easy to read.

When working the invention a group of probes for detecting a specific type or class of target molecule(s) may be provided with a common tag whereby the presence or amount of this type or class of target molecule(s) can be determined using said tag prior to, or possibly after, detecting individual members of the class using the distinctive barcode. Alternatively, a specific type of sample may be provided with a common tag whereby the detection of a particular target molecule(s) in the assay can be linked to a particular sample, for example, and without limitation, a particular tag may be used to designate a particular patient sample and the barcodes associated with the different probes may be used to detect different target molecule(s) found in or associated with that patient sample.

In some respects this tag or label can be viewed as a secondary barcoding system. The first identification sequence of nucleotides or barcoding region (typically between 18-5 nucleotides) is used to identify specific target molecule(s) whilst the secondary barcoding system is used to identify specific samples or groups/types of target molecule(s). For example, where specific samples are to be monitored, if 10 different serum samples are investigated in one study, we can combine all the PCR products into a single next generation sequencing run (greatly reducing cost), and the secondary barcoding will allow us, during sequence analysis, to identify the particular sample from which each specific target molecule(s) came.

In a further preferred embodiment of the invention said tag or label is attached to said probe at a site remote from said binding partner so as not to interfere with the binding function of same. Ideally said tag or label is incorporated into at least one of the primer sequences i) or ii) of the oligonucleotide b) of the probe of the invention. More preferably said tag or label is incorporated into both primer sequences i) or ii) of the oligonucleotide b) of the probe of the invention.

In a further preferred embodiment of the invention said first sequence is positioned nearest to said binding partner and said second sequence is positioned furthest away from said binding partner. Alternatively, said second sequence is positioned nearest to said binding partner and said first sequence is positioned furthest away from said binding partner.

In a further preferred embodiment of the invention said identification sequence of nucleotides or Barcode comprises, or consists of the following group of nucleotides, 18, 17, 16, 15, 14, 13, 12, 11, 10, 9, 8, 7, 6 or 5 nucleotides and, in any event, a number of nucleotides sufficient to provide the number of sequence combinations required to work the assay. For example 10 nucleotides provide for 1,048,576 combinations whereas 15 nucleotides provides for 1,073,741,824 combinations. Our current preferred design contains 16 nucleotides for 4,294,967,296 combinations.

In a further preferred embodiment of the invention for low-throughput applications, said barcode region may include or comprise at least one restriction enzyme site for the enzymatic cleavage of same, such as a BamH1 or HindIII site, although any other suitable restriction enzyme site known to those skilled in the art may be used.

In a further preferred embodiment of the invention said probe comprises single-stranded DNA, although double-stranded DNA may be used to provide stability, reduce non-specific interactions and alleviate potential steric hindrance.

According to a second aspect of the invention there is provided a plurality of probes for use in a multiplex assay to detect at least one target molecule(s) in at least one sample wherein each probe comprises:
a) at least one binding partner that is specific for at least one of said target molecule(s) and, attached thereto;
b) an oligonucleotide wherein said oligonucleotide comprises:
   i) a first sequence that is complementary to a forward primer sequence for amplification of said oligonucleotide;
   ii) a second sequence that is complementary to a reverse primer sequence for amplification of said oligonucleotide; and
   iii) positioned between said first and second sequences an identification sequence of nucleotides or Barcode wherein said Barcode acts an indicator for said target molecule(s) and consists of a certain number of nucleotides arranged in a unique order and further wherein the number of unique arrangements of said nucleotides provided by the number and nature of said nucleotides is greater than the number of target molecules in said sample; and
c) a tag or label that facilitates the identification of same in a multiplex, assay.

In a preferred embodiment of the invention said first and second sequences are common to a number, and ideally all, of the probes to facilitate the amplification of said oligonucleotide in the herein described method of the invention.

In yet another preferred embodiment of the invention at least two of said probes, and ideally more, are provided with different binding partners whereby a number of different target molecules(s) in at least one sample can be identified.

More preferably still, probes used to detect a specific type, or group of target molecules(s) are provided with a first common tag or label whereas probes used to detect another specific type or group of target molecules(s) are provided with a second common tag or label. Additionally or alternatively, probes used to identify a specific sample are provided with another common tag or label. Ideally, these tags or labels are short nucleotide sequences provided in at least one, or both, of the primer regions i) or ii) of the oligonucleotide b) of the probe of the invention. In this context short means 3-15 nucleotides long, ideally 9-11 nucleotides long including any one of 9, 10 or 11 nucleotides. The current preferred tag/label in our design is 10 nucleotides long.

According to a third aspect of the invention there is provided a method for detecting at least one target molecule(s) in a sample comprising:
1) exposing a test sample to at least one probe comprising:
a) at least one binding partner that is specific for said target molecule(s) and, attached thereto;
b) an oligonucleotide wherein said oligonucleotide comprises:
i) a first sequence that is complementary to a forward primer sequence for amplification of said oligonucleotide;
ii) a second sequence that is complementary to a reverse primer sequence for amplification of said oligonucleotide; and
iii) positioned between said first and second sequences an identification sequence of nucleotides or Barcode wherein said Barcode acts an indicator for said target molecule(s) and consists of a certain number of nucleotides arranged in a unique order and further wherein the number of unique arrangements of said nucleotides provided by the number and nature of said nucleotides is greater than the number of target molecules in said sample;
under conditions that enable said probe(s) to bind with said target molecule(s) to be detected to form at least one probe-target molecule(s) conjugate(s);
2) separating said conjugate(s) from said sample;
3) exposing said separated conjugate(s) to at least one forward and reverse primer pair, wherein one member of each pair is complementary to said first sequence in one of said probes and the other member of each pair is complementary to said second sequence in said same probe, and reagents suitable for performing polymerase chain reaction (PCR);
4) amplifying said oligonucleotide, using polymerase chain reaction (PCR); and
5) detecting said target molecule(s) in said sample by determining the presence of said amplified oligonucleotide.

According to a fourth aspect of the invention there is provided a multiplex method for detecting at least one target molecule(s) in at least one sample comprising:
1) exposing at least one test sample to a plurality of probes wherein each probe comprises:
a) at least one binding partner that is specific for at least one of said target molecule(s) and, attached thereto;
b) an oligonucleotide wherein said oligonucleotide comprises:
i) a first sequence that is complementary to a forward primer sequence for amplification of said oligonucleotide;
ii) a second sequence that is complementary to a reverse primer sequence for amplification of said oligonucleotide; and
iii) positioned between said first and second sequences an identification sequence of nucleotides or Barcode wherein said Barcode acts an indicator for said target molecule(s) and consists of a certain number of nucleotides arranged in a unique order and further wherein the number of unique arrangements of said nucleotides provided by the number and nature of said nucleotides is greater than the number of target molecules in said sample;
c) a tag or label that facilitates the identification of same in a multiplex assay;
under conditions that enable said probes to bind with said target molecule(s) to be detected to form probe-peptide conjugates;
2) separating said conjugates from said sample;
3) exposing said separated conjugates to at least one or a plurality of forward and reverse primer pairs, wherein one member of each pair is complementary to said first sequence in one of said probes and the other member of each pair is complementary to said second sequence in said same probe, and reagents suitable for performing polymerase chain reaction (PCR);
4) amplifying said oligonucleotides using polymerase chain reaction (PCR); and
5) detecting said target molecule(s) in said sample(s) by determining the presence of said amplified oligonucleotide and/or said tag.

In a preferred method of the invention separating said conjugates can be undertaken using any preferred laboratory technique such as washing, filtration, migration, precipitation, immuno-precipitation or centrifugation.

Ideally, immuno-precipitation is practiced where antibodies to the binding partner of the probe, or the peptide or protein to be detected, are used to selectively remove the conjugate(s) from the sample, ideally the antibodies are monoclonal, although polyclonal antibodies may also be used.

In a further preferred method of the invention detecting said target molecule(s) in said sample can be undertaken by sequencing said identification sequence of nucleotides or barcode; moreover, in the fourth aspect of the invention this can additionally or alternatively be undertaken by sequencing said tag.

In a further preferred method of the invention said sample is selected from the group comprising a sample of: blood; serum; semen; lymph fluid; cerebrospinal fluid; tears; saliva; urine; feces; tissue; and sweat. Alternatively, the sample may be an environmental sample such as water, soil or oil.

Those skilled in the art will know how to conduct polymerase chain reaction (PCR) to amplify said oligonucleotide.

Those skilled in the art will also appreciate that the specificity of the binding partner for its counterpart ensures the specificity of the assay and so eliminates non-specific binding or background noise, moreover, it also ensures specific binding at low concentrations and so where the size of the molecular signal is small. This feature, coupled with the PCR amplification step, ensures the small signal is detectable and so significantly increases the sensitivity of the assay. More advantageously still, the coupling of each probe with a tag ensures the results of the assay can be rapidly realized, thus increasing the efficiency of the system and lending it to high through-put screening. Additionally, the use of multiple probes within an assay method enables multiplex investigations and so enables one to determine whether a particular signal is present in multiple samples and/or whether a number of signals are present in either a single sample or multiple samples.

According to yet a further aspect of the invention there is provided a kit for detecting at least one target molecule(s) in at least one sample comprising: at least one probe or a library of probes in accordance with the invention, optionally, at least one primer pair for polymerase chain reaction (PCR) amplifying said probe and/or sequencing said probe and/or reagents or instructions pertaining thereto.

Those skilled in the art will appreciate that in so far as the probe is concerned the invention involves:
1) Linking of a specific binding partner (P=peptide or protein) which cannot be amplified with a molecule (O=oligonucleotide) which can be amplified;

2) Specific incorporation of a unique identifier or bar-code (BC) region in O so that a large number of P-O probes can be used in a single assay/tube; and ideally
3) Incorporation of an identification tag in one or both of the amplification primer regions of the oligonucleotide that will allow for the processing of multiple samples within a single high-throughput massively parallel sequencing reaction.

Those skilled in the art will also appreciate that the invention, advantageously, can be worked by creating probe libraries that are specific for particular lines of enquiry or investigation. Thus, for example, libraries can be created that include probes designed to detect selected pathogens, such as bacteria and viruses and, more advantageously probes that are designed to detect the immunodominant epitopes of said pathogens. Yet more particularly, libraries of probes can be created to detect pathogens known to cause specific diseases, such as, but not limited to, human encephalitis or respiratory diseases. Indeed, a library of probes may be created, containing, e.g. 100-150 P-O probes covering the major respiratory diseases. Further, libraries of probes can be created to undertake serological testing to determine, for example, the presence of enteroviruses.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The invention will now be described by way of example only with reference to the following figures:

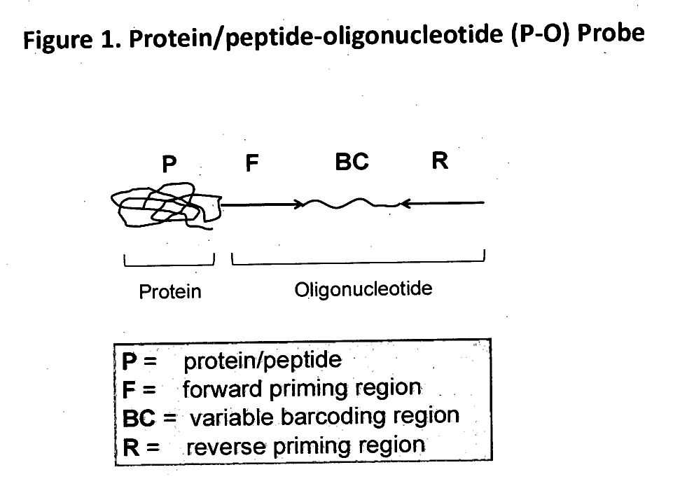

FIG. 1. Basic diagram to show the design of a generic P-O probe;

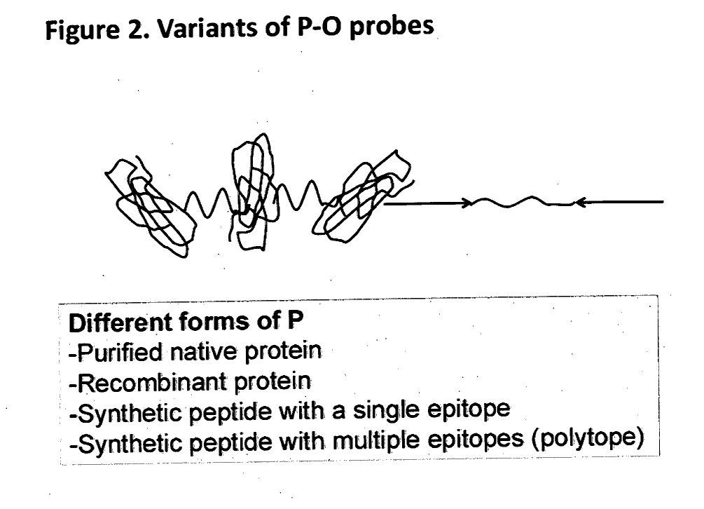

FIG. 2. Different forms of P (peptide or protein) that can be incorporated in this new platform;

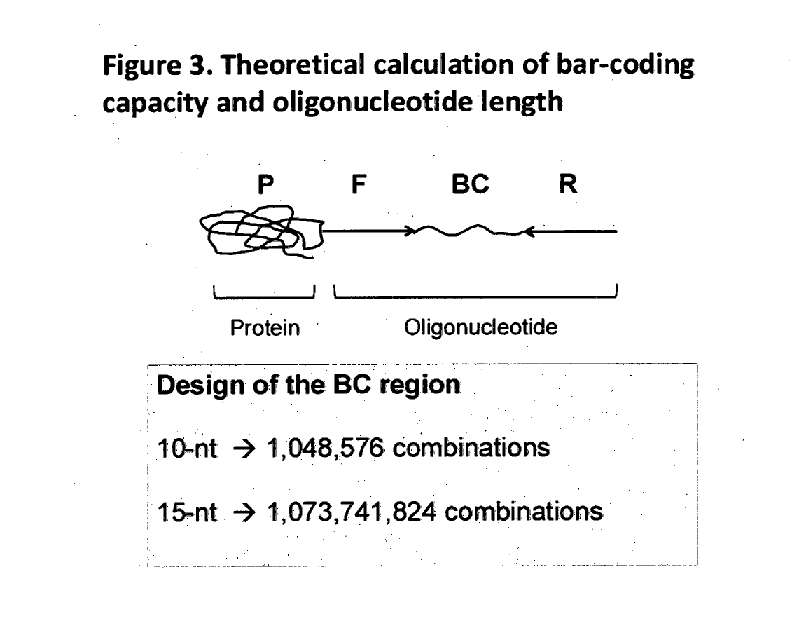

FIG. 3. Theoretical calculation of bar-coding capacity and oligonucleotide length;

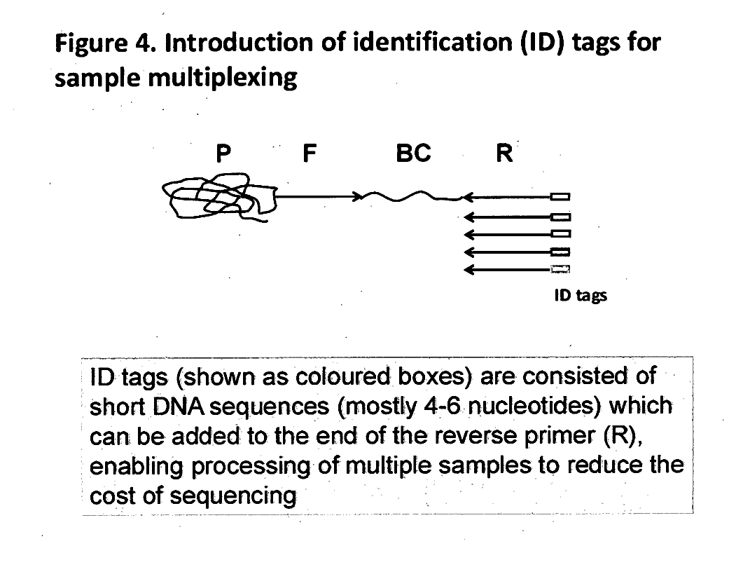

FIG. 4. Principle of incorporating an identification tag to enable processing of multiple samples in order to reduce cost and increase output;

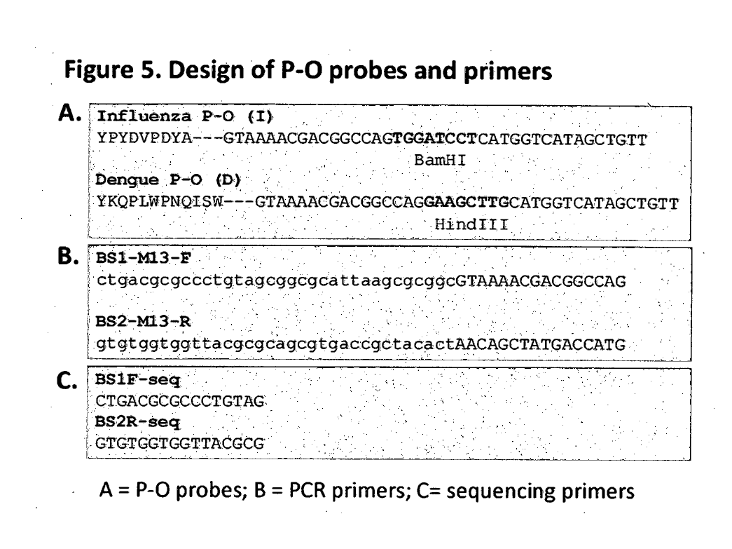

FIG. 5. shows the design of P-O probes and primers, in this example a restriction site is used as the barcode region;

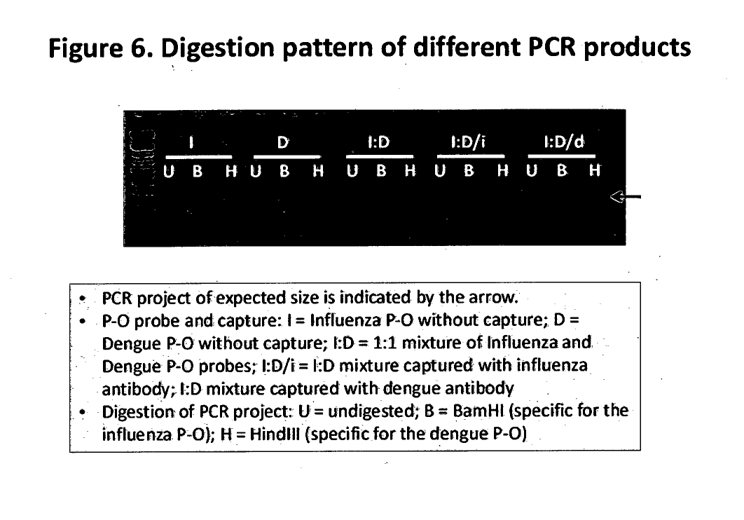

FIG. 6. shows the digestion pattern of different PCR products;

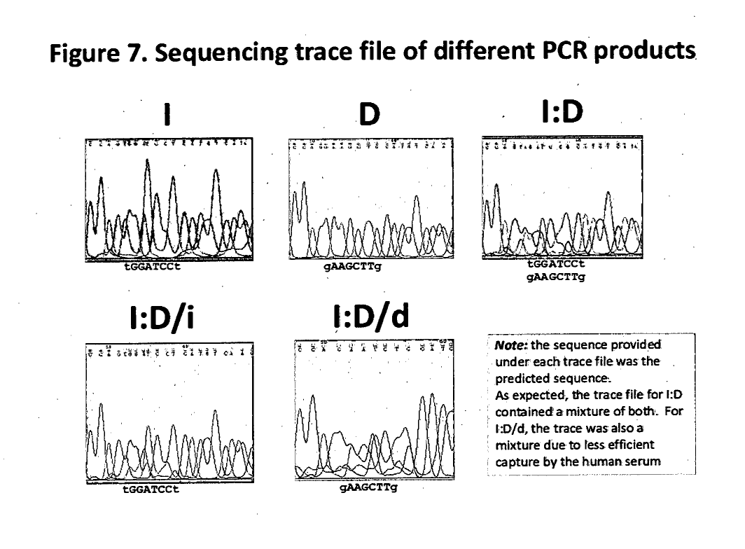

FIG. 7. shows the sequencing trace file of different PCR products;

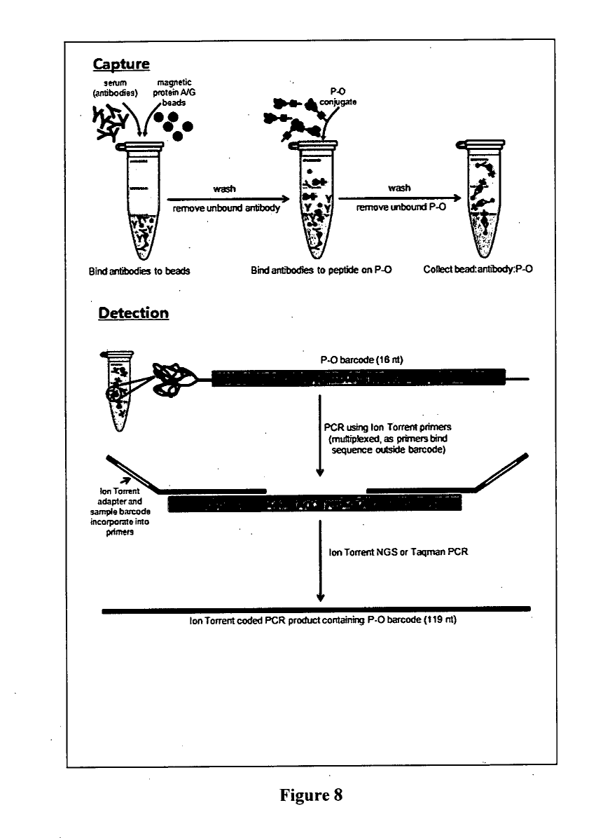

FIG. 8. shows schematically the MOST capture/detection procedure. The MOST procedure for the detection of specific antibodies in serum is dived into two parts, capture and detection. Step 1—Capture: Magnetic beads are placed into an eppendorf tube with the serum sample to be tested and incubated in a binding buffer to bind the antibodies to the Protein A/G magnetic beads. Following incubation, the magnetic beads are washed to remove any unbound antibody. The P-O conjugates are then added, again in binding buffer, to the eppendorf containing the magnetic beads. The peptide region of the P-O conjugate binds to its specific antibody during this incubation. Following incubation, the magnetic beads are washed to remove unbound P-O conjugates. Step 2—Detection: The magnetic beads are collected directly into a PCR mastermix that contains Ion Torrent specific primers. The P-O specific region of the Ion Torrent primer binds the sequence of the oligo located outside the P-O barcode. Each Ion Torrent primer set also contains a unique sample barcode, in addition to an adapter sequence. The captured oligos are amplified by PCR, then column purified to remove PCR reagents and magnetic beads. This sample is then analysed by Ion Torrent NGS. Although we have utilized the Ion Torrent platform in this example of the detection procedure, the application as a whole is not in any way dependent on the use of the Ion Torrent platform for resolution of results by next generation sequencing; other platforms are equally valid and can be used freely according to the individual platform specifications. The sample may also be monitored by Taqman quantitative PCR, where the Taqman probe is specific for the P-O barcode.

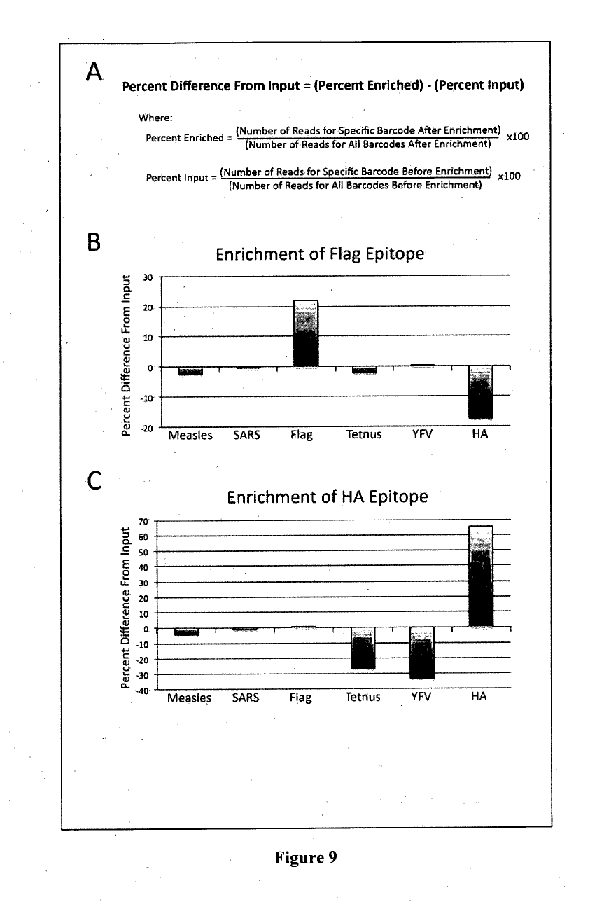

FIG. 9. shows Deep-sequencing results of MOST enrichment. Following treatment with MOST, samples were deep sequenced to determine the specific level of enrichment. To quantify enrichment, we subtracted the percent of reads specific to each barcoded target in the sample reaction before enrichment from the percent of reads specific to each barcoded target in the sample reaction after enrichment (A). This calculation highlights the target(s) that are enriched in the sample relative to the input as positive values and displays the target(s) that are diminished relative to the input as negative values. When human serum was spiked with 1 ul of anti-Flag antibody and subjected to MOST, the specific Flag signal increased over 21% relative to the input whereas the signal from the other P-O conjugates present in the reaction were unaffected or decreased relative to the input (B). When 5 ul of anti-HA antibody was used to simulate an immune response induced by influenza infection, the specific HA signal increased over 65% relative to the input whereas the signal from the other P-O conjugates present in the reaction were unaffected or decreased relative to the input (C); and

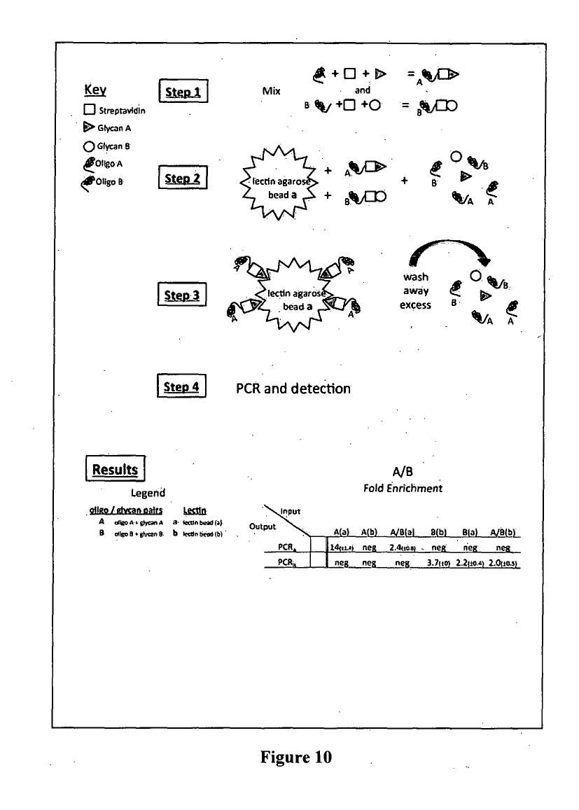

FIG. 10. shows Stepwise construction of the oligo:streptavidin:glycan complex. oligo A, glycan A and streptavidin are mixed together in one sample and oligo B, glycan B and streptavidin are mixed in another sample (Step 1). After a brief incubation period, these two samples are mixed and a lectin-agarose bead that is specific for only one of the glycans is added to the mixture to bind the specific oligo complex (Step 2). Multiple wash steps are implemented to deplete excess oligo A, oligo B, glycan A, and glycan B (Step 3) following which, the agarose bead-lectin:glycan:streptavidin:oligo complex is subjected to PCR and detection (Step 4). (Results) Fold enrichment. The reaction input is the final glycan:streptavidin:oligo complex that is made either with oligo A (A) or oligo B (B) or a mixture of both complexes oligo A/oligo B (NB). The binding of each lectin is specific for each glycan; lectin (a) should only bind glycan A and lectin (b) should only bind glycan B. The input Aa is then a glycanA:streptavidin:oligoA complex that is pulled out with lectin (a). A(b) is a glycanA:streptavidin:oligoA complex that is pulled out with lectin (b); the incorrect lectin for that glycan. NB(a) is therefore a mixture of glycanA:streptavidin:oligoA and glycanB:streptavidin:oligoB and the lectin specific for glycan A, lectin (a) is then used to pull out only the complex containing glycanA:streptavidin:oligoA while the glycanB:streptavidin:oligoB complex remains in solution and is washed away. B(b) is using lectin (b) to pull out glycanB:streptavidin:oligoB complex while B(a) is using the incorrect lectin (a) to attempt to pull out the glycanB:streptavidin:oligoB complex. A/B(b) is again the mixture of both complexes and using lectin (b) to capture only the glycanB:streptavidin:oligoB complex. After specific capture by the lectin-agarose bead, the oligo on the agarose-lectin:glycan:streptavidin:oligo bead complex is detected by TaqMan qPCR and the ΔCt or fold enriched is calculated relative to background. PCR_{A} is using a TaqMan qPCR probe designed to detect only oligo A, while PCR_{B} is a specific TaqMan qPCR probe designed to detect only oligo B. Once the PCR is performed and the Ct values are obtained, the ΔCt or fold enriched from background is calculated using 2^{(negative control Ct-output Ct)}.

### METHODS

Although the invention can be applied to all areas of biological research and development, we will use the monitoring of immune responses (antibodies) to illustrate the mode of practice.

### General Outline:

As shown in FIG. 1, each binding partner P specific for a target (peptide or protein) is covalently linked with an oligonuleotide (O) to form a P-O probe. Unlimited numbers of P-O probes can be mixed in an equal molar ratio, forming a library of P-O probes. When this library is examined with target peptide or protein such as antibodies (e.g., patient sera), specific binding will occur between the antibodies and their specific binding partners Ps. After capture and washing, PCR will be applied to amplify the BC region, followed by high-throughput massively parallel sequencing for identification and quantification of each BC.

Specific steps of practice:
1) Design/selection of binding partner P: As outlined in FIG. 2, there are multiple forms of binding partners i.e. peptides or proteins, which can be used in this platform. A polytope P, i.e. a binding partner that comprises a plurality of peptides or proteins and so epitopes, can be used to save cost, but may reduce specificity and should only be used when cost is of main concern. We envisage that a single peptide or protein P binding partner comprising one or more epitopes (as shown schematically in FIG. 1) will be the most likely form to use on a large scale as it provides the best sensitivity, epitope resolution and quality assurance.
2) Design of O: Any sequence can be used for the Forward and Reverse sites, some optimization makes sure that the most uniform amplification is achieved with the optimized F/R combination. The size of the barcode region will depend on the maximal multiplex envisaged (FIG. 3). We believe that a 10-nucleotide region (accommodating more than a million individual P-O probes) should be sufficient, although a shorter barcode region e.g. 5 nucleotides or a longer bar code region e.g. 15 nucleotides may be used. This shorter 10 nucleotide BC region, with respect to a larger 15 nucleotide BC region, may also favor unbiased amplification by the F/R primers.
3) Incorporation of an identification (ID) tag in the PCR primer region(s): An identification tag (typically 4-6 nt long) will be incorporated in at least one of the primer regions (or possibly both to increase reliability) so that multiple samples can be processed within the same sequencing reaction (see below) to reduce cost, FIG. 4.
4) Capture of specific P-O-peptide or protein conjugates by antibodies:

Although different methods can be used to capture specific peptide or protein and binding partner-P binding, we prefer to do this in liquid phase to increase the specificity (i.e., reduce background binding). Magnetic beads coated with, e.g. specific antibodies (e.g., anti-human IgG or anti-human IgM) are incubated with human serum first, followed by extensive washing. The P-O probe library will then be added to the antibody-bead mixture in a suitable buffer system. After incubation, the beads will be washed extensively to remove any unbound P-O probes.
5) PCR amplification: PCR reaction mixture (including primers, dNTPs and enzyme) will be added directly to the washed beads without any further treatment. The number of cycles for PCR amplification could vary but should generally be kept at a minimum to maintain the accuracy of peptide quantification.
6) Barcode readout: The identification and quantification of barcodes can be achieved using a variety of existing technologies known to those skilled in the art. High-throughput massively parallel sequencing (such as the Ion Torrent platform) can be used for “discovery” type of applications when more than 100 targets are investigated at the same time. For targets in the range of 10-100, it is possible to use Droplet Digital PCR (e.g., the BioRad system) and for samples with less than 10 targets, Luminex or qPCR can be applied for identification.

### Specific Examples

We herein demonstrate the working of the invention using two example probes/P-O and positive antibodies specific for each. The target protein. in this case was epitopes specific for influenza virus and denuge virus.

### P-O Conjugate and Primer Design (See FIG. 5):

1) Two specific peptide epitopes (YPYDVPDYA and YKQPLWPNQISW shown left hand side of FIG. 5 part A) were chosen: one from influenza virus and another from dengue virus
2) In this particular example, for the oligonucleotide design, a specific restriction enzyme site was incorporated into each one of the distinctive barcoding regions i.e. a different restriction site into each one so that we could use enzymatic digestion to corroborate the results from sequencing. BamH1 was incorporated into the barcoding region of the influenza specific probe and HindIII was incorporated into the barcoding region of the dengue specific probe.
3) In this particular example, an 8-nt barcoding region was used in this trial. The PCR amplification primers (B) for oligonucleotide amplification and the sequencing primers (C) for the unique bar code region sequencing are shown in FIG. 5. Notably, these primers were designed for this particular experiment and the invention is not to be limited thereby, rather these primers are exemplary of the invention. Other primers may be designed by those skilled in the art for different applications.

### Experimental Procedures

Antibodies Used in this Study
1) Anti-influenza (i) monoclonal antibody: HA-tag (c29F4) Rabbit mAb (Cell Signaling Technology Cat #3724S).
2) Anti-dengue (d) human serum: from an individual know to be infected twice with dengue virus

### Immunocapture

1) A mixture of diluted influenza and dengue (I:D) P-O probes FIG. 5 A (each at around 30,000 molecules/μl) was prepared.
2) 10 μl of I:D P-O probe mix was added to 5 μl of serum sample and 85 μl of IP buffer (25 mM Tris, 150 mM NaCl, pH 7.2) and the mix was incubated at room temperature, with agitation, for 30 min.
3) During incubation, Protein G beads (Pierce) i.e. an affinity matrix for the isolation and purification of immunoglobulins, were prepared as follows: 1 ml of IP buffer was added to beads. Beads were then centrifuged at 2500×g for 2 min and supernatant was removed. This step was repeated twice, and beads were then resuspended in 500 μl of IP buffer.
4) Following incubation, 50 μl of beads were added to serum/P-O mix. The serum/P-O/bead solution was incubated for a further 30 min, at room temperature, with agitation.
5) After the incubation, 500 μl of IP buffer were added and beads were centrifuged for 2 min at 2500×g. Supernatant was removed and 1 ml of IP buffer was added to beads. This step was repeated 3 times.
6) Beads were resuspended in 39 μl of water and transferred to a PCR tube for direct use in the PCR.

### PCR Reactions

1) A 50 μl PCR reaction with set up directly in the tube containing the beads from the immunocapture. The 50 μl PCR reaction contained 5 μl of 10× buffer, 4 μl of 2.5 mM dNTPs, 1 μl of each primer (BS-M13F and BS-M13R) and 0.2 μl Atlas Taq polymerase.
2) The PCR cycling conditions for 40 cycles were as follows: denaturing at 94 C for 10 sec, annealing at 54 C for 10 sec and extension at 72 C for 15 sec.
3) PCR products were resolved on a 2% agarose gel.

### Restriction Enzyme Digestion

1) PCR products were purified using QIAquick PCR purification kit (Qiagen) according to manufacturer's instructions. Purified PCR products were eluted in 30 μl TE buffer.
2) A 30 μl digestion mixture was set up with either BamHI or HindIII. The digestion reaction contained 3 μl purified PCR product, 3 μl 10× buffer, 3 μl 10×BSA, 0.5 μl restriction enzyme and 20.5 μl H₂O.
3) Digestion reactions were incubated at 37° C. for 2 hrs then resolved on a 2% agarose gel.

### Sequencing

1) For Sanger sequencing, the purified PCR product was sent to an external service provider for Sanger sequencing using primers BS1 F and BS2R listed in FIG. 5.

### Deep Sequencing

We have also designed the oligo portion of the P-O conjugate so that the products of the PCR enrichment step are immediately ready for quantification by deep sequencing. In addition, we have also developed a qPCR assay that has the ability to discriminate and detect the bar-coding region of our probe(s). Deep sequencing analysis typically will be used primarily for high throughput screening of samples whereas qPCR is more likely to be the platform of choice for low-throughput applications of our technology. These new methodologies are outlined in FIG. 8 and are described in detail here:
Step 1—Capture: Magnetic Protein NG beads are placed into an eppendorf tube with 200 μl of block/binding buffer (1% blocking reagent [Roche #11 096 176 001] in 1×TBS-T [0.05% Tween], 0.1 mg/ml BSA, 100 μg/ml final conc tRNA). Although we have utilized the blocking buffer from Roche in this example of the capture procedure, the application as a whole is not in any way dependent on the use of the Roche blocking buffer in particular for the binding of construct to its specific target; other blocking buffers are likely to be equally valid and can be used freely according to the individual reagents specifications. A serum sample (or in the pilot study monoclonal antibodies), is added and incubated to bind antibodies present to the magnetic beads. Following incubation, the magnetic beads are washed in 500 μl 1×TBS-T (0.05% Tween) to remove any unbound antibody. The P-O conjugates (a mixture of P-O conjugate probes i.e. probes 1-6) are then added, again in 200 μl binding buffer, to the eppendorf containing the magnetic bead:antibody complex. The peptide region of the P-O conjugate binds to its specific antibody during this incubation. Following incubation, the magnetic beads are washed in 500 μl 1×TBS-T (0.05% Tween) to remove unbound P-O probes.

Step 2—Detection: The magnetic beads are collected directly into a 50 μl PCR mastermix that contains Ion Torrent specific primers. PCRs are performed with Pfu proofreading polymerase. The P-O specific region of the Ion Torrent primer binds the 18nt primer complementary sequences of the probes located outside the P-O barcode. This 18nt sequence on either side of the P-O barcode is typically identical for all P-O conjugates, allowing a multiplexed Ion Torrent PCR. Most preferably, each Ion Torrent primer set also contains a unique tag or label sequence, in addition to an adapter sequence. The captured oligos are amplified by PCR, and then column purified to remove PCR reagents and magnetic beads. The purified PCR product is eluted in 10 μl and the quality and quantity of the DNA is interrogated on a bioanalyzer DNA 1000 Chip. This sample is then analysed by Ion Torrent NGS. The sample may also be monitored by Taqman quantitative PCR, where the Taqman probe is specific for the P-O barcode.

### Exemplary Probes

In addition to the probes shown in FIG. 5, we have created an additional six constructs for working the technology. The peptide and oligonucleotide sequences for these constructs are shown in the table below.

| P-O Name | Oligo Sequence 5′-3′ (Barcode in uppercase) | Peptide |
|---|---|---|
| Oligo-001 | gagatagactcaagaccgACTCTACCGTCTACTActcatcaacttcgcatgg | DYKDDDDK |
| (Flag) | | |
| Oligo-002 | gagatagactcaagaccgACCAGATTCTGAAGTGctcatcaacttcgcatgg | YPYDVPDYA |
| (HA) | | |
| Oligo-003 | gagatagactcaagaccgCCAACGTCCCATCTGActcatcaacttcgcatgg | IIVGRGDSRLTY |
| (YFV) | | |
| Oligo-004 | gagatagactcaagaccgTCAGTACTTTCAGAATctcatcaacttcgcatgg | QYIKANSKFIGITEL |
| (Tetanus) | | |
| Oligo-005 | gagatagactcaagaccgTTCGGGCGGTCTCAATctcatcaacttcgcatgg | LTPAWR |
| (SARS) | | |
| Oligo-006 | gagatagactcaagaccgCTGACACGTACACCTTctcatcaacttcgcatgg | AEPLLSC |
| (MeV) | | |

### Results (see FIGS. 6 and 7)

1) Verification of barcoding sequence and functionality of the P-O probe: two different methods, restriction digestion and direct sequencing, were used to confirm the correct sequence of the barcoding region in each of the two P-O probes. As shown in FIGS. 6 and 7, the P-O probes contained the expected sequences and could be cut using BamHI and HindII, respectively.
3) Specific capture by cognate antibodies: to test the concept of specific capture by antibodies, a 1:1 mixture of I:D P-O probes were incubated with two different antibodies. Anti-influenza (i) monoclonal antibody: HA-tag (c29 F4) Rabbit mAb and an anti-dengue (d) human serum.

In each case, a specific enrichment was observed thus illustrating the functionality of the P-O probe design.

### Results (see FIGS. 9 and 10)

The results of quantification by deep sequencing are presented in FIG. 9. These results show that under the conditions described above, we are able to enrich the Flag epitope P-O conjugate and the influenza HA epitope conjugate by over 21% and 65% respectively.

Whilst the above information exemplifies the technology using a protein or peptide in a sample and thus a probe with a protein or peptide binding partner specific for the protein or peptide in the sample, we have also worked the invention using a glycan/carbohydrate in a sample and thus a probe with a binding partner specific for the glycan/carbohydrate in the sample. In FIG. 10, we show data for the glycan-oligo concept using a modified, indirect conjugation method employing biotin-streptavidin binding. These data show that using this methodology, we can enrich and detect specific glycans up to 14-fold relative to the negative control.

### CONCLUSION

1) Functionality of P-O probes: the trial confirmed that it is possible to conjugate a peptide or protein binding partner P with an oligonucleotide O and maintain the functionality of both entities
2) Functionality of barcoding: the concept of barcoding was proven to be effective in that we can easily identify influenza- or dengue-specific binding.
3) Quantification of binding: from the digest patterns and sequencing trace file, it is clear that an estimation of the enrichment factor can be obtained (FIG. 6 or 7, comparing I:D vs I:D/I and is actually shown in FIGS. 9 and 10). Based on these data, it is expected that accurate quantification can be easily achieved using NGS.
4) Optimization: the monoclonal antibody performed better than the polyclonal human serum, but it is expected that optimization of the primer design, PCR conditions or immunocapture should enhance performance.
5) Secondary barcoding: provides a way of further refining the technology.

1. A probe for detecting and/or quantifying at least one target molecule(s) in a sample comprising:
a) at least one binding partner that is specific for one of said target molecules(s) and, attached thereto;
b) an oligonucleotide wherein said oligonucleotide comprises:
i) a first sequence that is complementary to a forward primer sequence for amplification of said oligonucleotide;
ii) a second sequence that is complementary to a reverse primer sequence for amplification of said oligonucleotide; and
iii) positioned between said first and second sequences is an identification sequence of nucleotides or barcode wherein said barcode acts an indicator for said target molecule(s) and consists of a certain number of nucleotides arranged in a unique order and further wherein the number of unique arrangements of said nucleotides provided by the number and nature of said nucleotides is greater than the number of target molecules in said sample.

2. The probe according to claim 1 wherein said binding partner has a plurality of epitopes that are specific for said target molecule(s).

3. The probe according to claim 1 wherein said binding partner itself comprises at least one peptide and/or protein which comprises at least one epitope that is specific for said target molecule(s) to be detected.

4. The probe according to claim 3 wherein said binding partner comprises a plurality of epitopes.

5. The probe according to claim 1 wherein said identification sequence of nucleotides or barcode comprises a number of nucleotides selected from the group consisting of 18, 17, 16, 15, 14, 13, 12, 11, 10, 9, 8, 7, 6, and 5 nucleotides.

6. The probe according to claim 1 wherein said identification sequence of nucleotides or barcode includes or comprises at least one restriction enzyme site for the cleavage of same.

7. The probe according to claim 1 wherein said probe is further provided with at least one further tag or label that facilitates the identification of same in a multiplex assay.

8. The probe according to claim 7 wherein said tag or label is amplifiable by PCR and so comprises a short DNA sequence.

9. The probe according to claim 7 wherein said tag or label is attached to said probe at a site remote from said binding partner.

10. The probe according to claim 9 wherein said tag or label is incorporated into at least one of the said primer sequences i) or ii) of the oligonucleotide b).

11. The probe according to claim 1 wherein said target molecule is selected from the group comprising: a peptide, protein, lipid or carbohydrate.

12. A plurality of probes for use in a multiplex assay to detect at least one target molecule(s) in at least one sample wherein each probe comprises:
a) at least one binding partner that is specific for at least one of said target molecule(s) and, attached thereto;
b) an oligonucleotide wherein said oligonucleotide comprises;
i) a first sequence that is complementary to a forward primer sequence for amplification of said oligonucleotide;
ii) a second sequence that is complementary to a reverse primer sequence for amplification of said oligonucleotide; and
iii) positioned between said first and second sequences an identification sequence of nucleotides or barcode wherein said barcode acts an indicator for said target molecule(s) and consists of a certain number of nucleotides arranged in a unique order and further wherein the number of unique arrangements of said nucleotides provided by the number and nature of said nucleotides is greater than the number of target molecules in said sample; and
c) a tag or label that facilitates the identification of same in a multiplex assay.

13. The plurality of probes according to claim 12 wherein said first and second sequences are common to a number of the probes.

14. The plurality of probes according to claim 12 wherein at least two of said probes are provided with different binding partners whereby a number of different target molecule(s) in at least one sample can be identified.

15. The plurality of probes according to claim 12 wherein probes used to detect a specific type or group of target molecule(s) are provided with a first common said tag or label; or probes used to detect another specific type or group of target molecule(s) are provided with a second common said tag or label; or probes used to identify a specific sample are provided with another common said tag or label.

16. The plurality of probes according to claim 15 wherein said tags or labels are short nucleotide sequences provided in at least one, or both, of the primer regions i) or ii) of the oligonucleotide b).

17. The plurality of probes according to claim 12 wherein said target molecule is selected from the group comprising: a peptide, protein, lipid or carbohydrate.

18. A method for detecting at least one target molecule(s) in a sample comprising:
1) exposing a test sample to at least one probe comprising:
a) at least one binding partner that is specific for said target molecule(s) and, attached thereto;
b) an oligonucleotide wherein said oligonucleotide comprises:
i) a first sequence that is complementary to a forward primer sequence for amplification of said oligonucleotide;
ii) a second sequence that is complementary to a reverse primer sequence for amplification of said oligonucleotide; and
iii) positioned between said first and second sequences an identification sequence of nucleotides or barcode wherein said barcode acts an indicator for said target molecule(s) and consists of a certain number of nucleotides arranged in a unique order and wherein the number of unique arrangements of said nucleotides provided by the number and nature of said nucleotides is greater than the number of target molecules in said sample;
under conditions that enable said probe(s) to bind with said target molecule(s) to be detected to form at least one probe-target molecule(s) conjugate(s);
2) separating said conjugate(s) from said sample;
3) exposing said separated conjugate(s) to at least one forward and reverse primer pair, one of which is complementary to said first sequence and the other of which is complementary to said second sequence, and reagents suitable for performing PCR;
4) amplifying said oligonucleotide using PCR; and
5) detecting said target molecule(s) in said sample by determining the presence of said amplified oligonucleotide.

19. A multiplex method for detecting at least one target molecule(s) in at least one sample comprising:
1) exposing at least one test sample to a plurality of probes wherein each probe comprises:
a) at least one binding partner that is specific for at least one of said target molecule(s) and, attached thereto;
b) an oligonucleotide wherein said oligonucleotide comprises:
i) a first sequence that is complementary to a forward primer sequence for amplification of said oligonucleotide;
ii) a second sequence that is complementary to a reverse primer sequence for amplification of said oligonucleotide; and
iii) positioned between said first and second sequences an identification sequence of nucleotides or barcode wherein said barcode acts an indicator for said target molecule(s) and consists of a certain number of nucleotides arranged in a unique order and further wherein the number of unique arrangements of said nucleotides provided by the number and nature of said nucleotides is greater than the number of target molecules in said sample;
c) a tag or label that facilitates the identification of same in a multiplex assay;
under conditions that enable said probes to bind with said target molecule(s) to be detected to form probe-target molecule(s) conjugates;
2) separating said conjugates from said sample;
3) exposing said separated conjugates to at least one or a plurality of forward and reverse primer pairs, wherein one member of each pair is complementary to said first sequence in one of said probes and the other member of each pair is complementary to said second sequence in said same probe, and reagents suitable for performing PCR;
4) amplifying said oligonucleotides using PCR; and
5) detecting said target molecule(s) in said sample by determining the presence of said amplified oligonucleotide and/or said tag.

20. The method according to claim 18 or 19 wherein separating said conjugates can be undertaken using at least one technique selected from the following: filtration, migration, precipitation, immuno-precipitation and centrifugation.

21. The method according to claim 19 wherein detecting said peptide or protein in said sample is undertaken by sequencing said identification sequence of nucleotides or barcode and/or sequencing said tag.

22. The method according to claim 19 wherein said sample is selected from the group comprising: blood; serum; semen; lymph fluid; cerebrospinal fluid; tears; saliva; urine; tissue; sweat; water, soil and oil.

23. The method according to claim 19 wherein said target molecule is selected from the group comprising: a peptide, protein, lipid or carbohydrate.

24. A kit for detecting at least one target molecule(s) in at least one sample comprising:
i) at least one probe according to claim 1 and/or a plurality of probes according to claim;
ii) optionally, primer pairs for PCR amplifying said probe and/or sequencing said probe; and
iii) reagents and/or instructions pertaining thereto.

25. (canceled)


SEQUENCE LISTING (20 sequences)

SEQ ID NO: 1 - protein, 9 aa, artificial sequence
  Peptide Epitopes
YPYDVPDYA

SEQ ID NO: 2 - protein, 12 aa, artificial sequence
  Peptide epitope
YKQPLWPNQISW

SEQ ID NO: 3 - DNA, 52 bp, artificial sequence
  Probes Oligo-001
gagatagactcaagaccgactctaccgtctactactcatcaacttcgcatgg

SEQ ID NO: 4 - DNA, 52 bp, artificial sequence
  Probes Oligo-002
gagatagactcaagaccgaccagattctgaagtgctcatcaacttcgcatgg

SEQ ID NO: 5 - DNA, 52 bp, artificial sequence
  Probes Oligo-003
gagatagactcaagaccgccaacgtcccatctgactcatcaacttcgcatgg

SEQ ID NO: 6 - DNA, 52 bp, artificial sequence
  Probes Oligo-004
gagatagactcaagaccgtcagtactttcagaatctcatcaacttcgcatgg

SEQ ID NO: 7 - DNA, 52 bp, artificial sequence
  Probes Oligo-005
gagatagactcaagaccgttcgggcggtctcaatctcatcaacttcgcatgg

SEQ ID NO: 8 - DNA, 52 bp, artificial sequence
  Probes Oligo-006
gagatagactcaagaccgctgacacgtacaccttctcatcaacttcgcatgg

SEQ ID NO: 9 - protein, 8 aa, artificial sequence
  Probes
DYKDDDDK

SEQ ID NO: 10 - protein, 9 aa, artificial sequence
  Probes
YPYDVPDYA

SEQ ID NO: 11 - protein, 12 aa, artificial sequence
  Probes
IIVGRGDSRLTY

SEQ ID NO: 12 - protein, 15 aa, artificial sequence
  Probes
QYIKANSKFIGITEL

SEQ ID NO: 13 - protein, 6 aa, artificial sequence
  Probes
LTPAWR

SEQ ID NO: 14 - protein, 7 aa, artificial sequence
  Probes
AEPLLSC

SEQ ID NO: 15 - DNA, 40 bp, artificial sequence
  Probes P-O(I)
gtaaaacgacggccagtggatcctcatggtcatagctgtt

SEQ ID NO: 16 - DNA, 40 bp, artificial sequence
  Probes P-O (D)
gtaaaacgacggccaggaagcttgcatggtcatagctgtt

SEQ ID NO: 17 - DNA, 50 bp, artificial sequence
  Primer BS1-M13-F
ctgacgcgccctgtagcggcgcattaagcgcggcgtaaaacgacggccag

SEQ ID NO: 18 - DNA, 50 bp, artificial sequence
  Primer BS2-M13-R
gtgtggtggttacgcgcagcgtgaccgctacactaacagctatgaccatg

SEQ ID NO: 19 - DNA, 16 bp, artificial sequence
  Primer BS1F-seq
ctgacgcgccctgtag

SEQ ID NO: 20 - DNA, 16 bp, artificial sequence
  Primer BS2R-seq
gtgtggtggttacgcg